(19) United States
(12) Patent Application Publication
(10) Pub. No.: US 2025/0169908 A1
(43) Pub. Date: May 29, 2025

(54) MOLDED COMPONENTS FOR LONGITUDINAL MEMBER SUPPORT

(71) Applicant: CANON U.S.A., INC., MELVILLE, NY (US)

(72) Inventor: Inderpal Singh, Norwood, MA (US)

(21) Appl. No.: 18/958,412

(22) Filed: Nov. 25, 2024

Related U.S. Application Data
(60) Provisional application No. 63/603,513, filed on Nov. 28, 2023.

(51) Int. Cl. A61B 34/00; A61B 34/30; A61M 25/01

(52) U.S. Cl. CPC A61B 34/71; A61B 2034/301; A61M 25/0147

(57) ABSTRACT

Disclosed are a multi-part body for supporting a plurality of longitudinal members for operation of a continuum robot, the body including a first part, a first plurality of nodes formed on or through the first part and surrounding a longitudinal axis; a second part; a second plurality of nodes formed on or through the second part and surrounding the longitudinal axis; and grooves formed along a longitudinal axis by joining the first part to the second part and aligning the first plurality of nodes with the second plurality of nodes.

### CROSS-REFERENCE TO RELATED APPLICATION(S)

This application claims the priority under 35 U.S.C. § 119(e) to U.S. Provisional Application No. 63/603,513, which was filed on Nov. 28, 2023, the entire disclosure of which is incorporated herein by reference.

### FIELD OF THE DISCLOSURE

The present disclosure relates generally to continuum robots applicable to guide devices, including medical devices, interventional tools, instruments, and endoscopes.

### BACKGROUND OF THE DISCLOSURE

A continuum robot (also referred to as a snake) includes a plurality of bending sections having a flexible structure, with the shape of the continuum robot being controlled by deforming the bending sections. The snake has significant advantages over existing robots including rigid link robots. An advantage is that the snake can move along a curve in a narrow space or in an environment with scattered objects in which the rigid link robot may get stuck. Another advantage is that it is possible to operate the snake without damaging surrounding fragile elements, utilizing intrinsic flexibility of the snake.

In recent years, minimally invasive medical care, with which burden on the patient can be reduced and quality of life after treatment or inspection can be improved, has been attracting attention. A surgery or inspection using an endoscope is a typical example of minimally invasive medical care. For example, a laparoscopic surgery is advantageous over a conventional abdominal surgery in that it can be performed with a smaller surgical wound, which results in a shorter stay in the hospital and less damage to the appearance.

Endoscopes used for the minimally invasive medical care are roughly divided into rigid endoscopes and soft endoscopes. Although a rigid endoscope may provide clear images, the direction in which an observation target can be observed is limited. In addition, when the rigid endoscope is inserted into a curved organ, e.g., esophagus, large intestine, or urethra, an insertion portion of the rigid endoscope may press on the organ. In contrast, a soft endoscope includes an insertion portion formed of a bendable member, so that a large area can be observed in detail by adjusting the bending angle of the distal end of the endoscope. In addition, by bending the insertion portion along an insertion path, burden on the patient can be reduced. When the number of bendable portions is increased, the endoscope can be inserted to a deep area of the body without causing the endoscope to come into contact with tissue even when the insertion path has a complex curved shape.

Accordingly, soft endoscopes having a plurality of bendable portions have been researched and developed.

Various related art disclosures in the field include U.S. Pat. No. 11,559,190, which discusses a steerable device with push-pull actuators and breakout unit, as well as WO 2022/146751 which discusses a steerable snake with push-pull rod structure. U.S. 2022/0202277 discusses a medical apparatus having a bendable body with a driving wire; a break-out wire attached to the driving wire, with a distal end of the break-out wire attached to a proximal end of the driving wire; a distal guide tube guiding the driving wire and ending before the break-out wire with a space; a resilient element abutting the driving wire along at least a portion of a longitudinal direction of the driving wire; and an actuator configured to retract and advance the driving wire via the break-out wire thereby maneuvering the bendable body. Each of the afore-mentioned disclosures are incorporated herein by reference.

When controlling the bendable medical device by pushing or pulling the small diameter drive wires, the amount of operating force that can be applied to the drive wires is limited by a buckling force of the specific wire diameter and material. For the bendable medical device, space constraints imposed by target anatomy, tool dimensions, and the like, required use of small diameter wires. To prevent wire buckling, continuous support may be provided around the drive wires throughout the entire length of the bendable medical device. U.S. 2015/0142013 discusses releasing tension from the continuum robot pull wires with a button/command for the continuum robot shape to conform to the anatomy. U.S. 2019/0105468 also discusses problematic buckling, especially as the size/diameter of the snake robot is decreased. When controlling the bendable medical device by pushing/pulling small diameter drive wires, the amount of operating force that can be applied to the drive wires is limited by wire buckling. Use of small diameter wires is important in bendable medical devices due to space constraints from the target anatomy, tool dimensions, etc. To prevent wire buckling, continuous support around the drive wires throughout an entire length of the bendable medical device is desired.

### SUMMARY

Thus, an aspect of the present disclosure provides a multi-part body for supporting a plurality of longitudinal members, the multi-part body including a first part; a first plurality of nodes formed on or through the first part, surrounding a longitudinal axis; a second part; a second plurality of nodes formed on or through the second part, surrounding the longitudinal axis; and a plurality of grooves formed along a longitudinal axis by joining the first part to the second part and aligning the first plurality of nodes with the second plurality of nodes.

Another aspect of the present disclosure provides a longitudinal support formed by alignment of multi-part bodies, with the longitudinal support including a plurality of multi-part bodies arranged in series along a longitudinal axis, each including a first part with a first plurality of nodes, a second part with a second plurality of nodes. Each first plurality of nodes is formed on or through the respective first part, surrounding the longitudinal axis. Each second plurality of nodes is formed on or through the respective second part, surrounding the longitudinal axis. A plurality of grooves are formed along the longitudinal axis by alignment of respective first and second plurality of nodes of the plurality of multi-part bodies.

A further aspect of the present disclosure provides a connector that includes at least one body including a first part with a first plurality of nodes, a second part with a second plurality of nodes, and a plurality of grooves; a hub; and a plurality of longitudinal members. The at least one body is formed by joining the first part to the second part. The first plurality of nodes are formed on or through the first part, positioned along a periphery of the first part around a longitudinal axis. The second plurality of nodes are formed on or through the second part positioned along a periphery of the second part around the longitudinal axis. The plurality of grooves are formed by aligning the first plurality of nodes with the second plurality of nodes. Each longitudinal member of the plurality of longitudinal members is supported in a respective groove of the plurality of grooves.

These and other objects, features, and advantages of the present disclosure will become apparent upon reading the following detailed description of exemplary embodiments of the present disclosure, when taken in conjunction with the appended drawings, and provided paragraphs.

### BRIEF DESCRIPTION OF THE DRAWINGS

Further objects, features and advantages of the present innovation will become apparent from the following detailed description when taken in conjunction with the accompanying figures showing illustrative embodiments of the present innovation.

FIG. 1 is a block diagram illustrating components of a system for operation of a continuum robot.

FIG. 2 illustrates components of the continuum robot.

FIG. 3 illustrates an actuator and hub assembled onto a shaft of the continuum robot.

FIG. 4 is a perspective view of components within a hub assembly and related components.

FIG. 5 is a profile view illustrating components within the catheter hub and related components.

FIG. 6 is perspective view of the components within the catheter hub and related components.

FIG. 7 is a perspective view illustrating connection of proximal components of the catheter hub affixed in respective clamps of a controller.

FIG. 8A is a side view of a body supporting a longitudinal member, according to the present disclosure.

FIG. 8B is a perspective view of the body of FIG. 8A, according to the present disclosure.

FIG. 8C is a sectional view of the body of FIG. 8A, according to the present disclosure.

FIG. 9 is a side view of a plurality of bodies providing multiple contact support points for each longitudinal member, according to the present disclosure.

FIG. 10 is a side view of a plurality of bodies supporting a longitudinal member, according to the present disclosure.

FIGS. 11A to 11D are side views of a first part and a second part of a multi-part body, according to the present disclosure.

FIGS. 12A to 12D illustrate various parting line positions and varied sizing of the first part and the second part of the multi-part body, according to the present disclosure.

FIG. 12E is a side view of a first part with an extended length, according to the present disclosure.

Throughout the figures, the same reference numerals and characters, unless otherwise stated, are used to denote like features, elements, components or portions of the illustrated embodiments. Moreover, while the subject disclosure will now be described in detail with reference to the figures, it is done so in connection with the illustrative exemplary embodiments. It is intended that changes and modifications can be made to the described exemplary embodiments without departing from the true scope and spirit of the subject disclosure as defined by the appended claims.

### DETAILED DESCRIPTION

The resent disclosure has several embodiments and relies on patents, patent applications and other references for details known to those of the art. Therefore, when a patent, patent application, or other reference is cited or repeated herein, it should be understood that it is incorporated by reference in its entirety for all purposes as well as for the proposition that is recited.

In the subject disclosure, systems and mechanisms of a continuum robot are described, followed by continuum robot support elements for reducing buckling, as well as the systems and procedures associated with the continuum robot and said support elements.

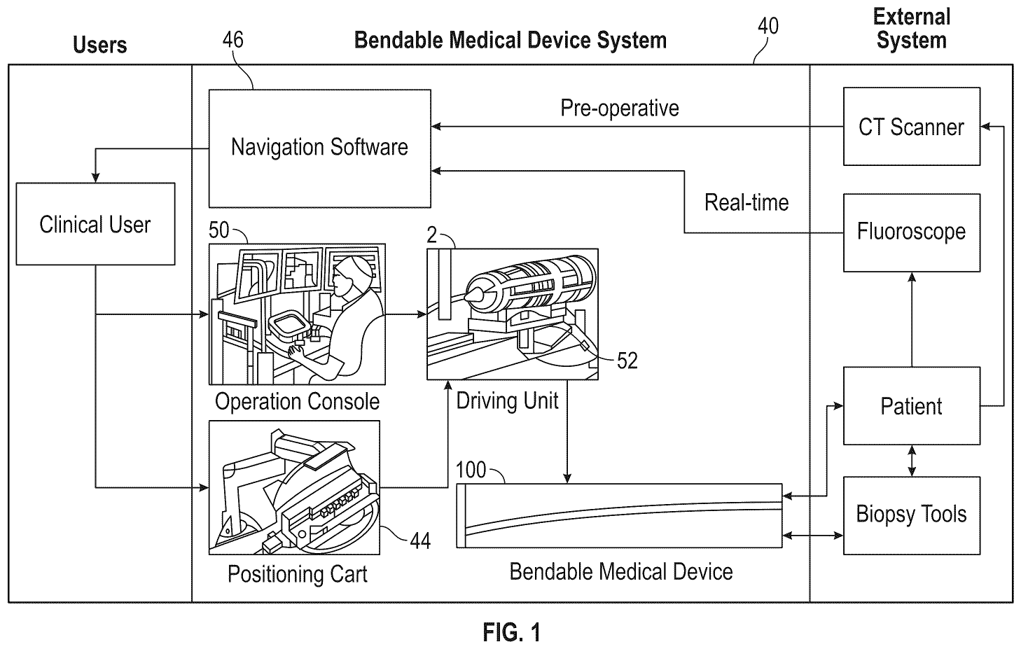

FIG. 1 is a block diagram illustrating components of a system for operation of a continuum robot.

As illustrated in FIG. 1, the system 40 includes a driving unit 2 (also referred to as an actuator or driver) for driving the drive wires, and having a base stage 52, a continuum robot 100 (also referred to as bendable medical device, steerable catheter, snake, or robotic catheter), a positioning cart 44, an operation console 50 (also referred to as controller or control system), having push-button, thumb-stick, and/or joystick, and navigation software 46. The medical device system 40 is capable of interacting with external system components and clinical users to facilitate use in a patient.

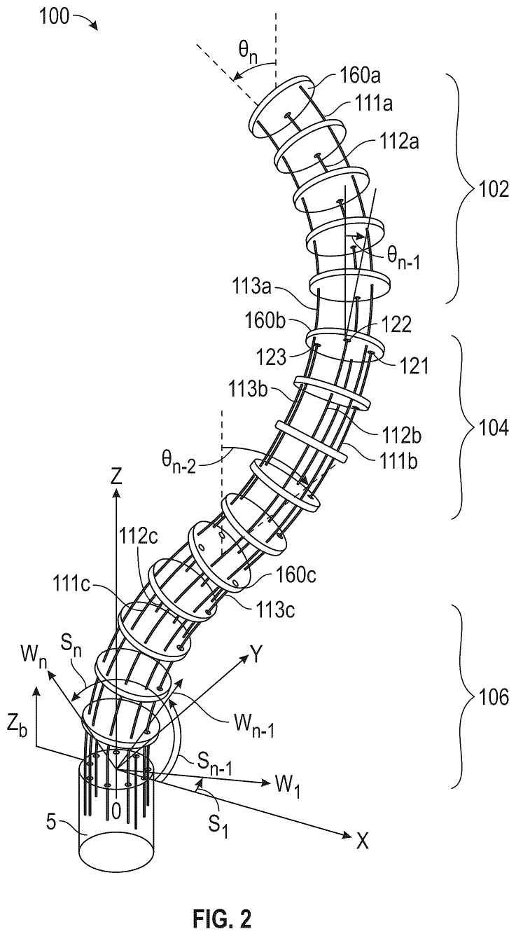

FIG. 2 illustrates components of a continuum robot.

As shown in FIG. 2, the continuum robot 100 includes push/pull drive wires 111b, 112b and 113b, which are connected to connection portions 121, 122 and 123, respectively, found on an end disc 160b, for controlling the middle bending section 104. Additional drive wires (three for each of the other bendable sections 102 and 106) 111a, 111c, 112a, 112c, 113a, 113c, are attached at the distal ends of each bendable section 102 and 106, to the respective end disc 160a and 160c. Each bending section is operated similarly. Thus, the description of one bending section, i.e., the middle bending section 104, will be recognized to apply to the other sections. Posture of the bending section 104 is controlled by pushing and pulling the wires 111b to 113b by using actuators, with I_{d}=the length of the central axis a bending section; θₙ=the bending angle of the distal end; ξₙ=the rotational angle of the distal end; ρₙ=the radius of curvature of a bending section.

The continuum robot 100 attaches to a catheter shaft 5, which may be disposed on the base stage 52 and may be moved by the base stage 52 in the longitudinal direction, to advance, retard and/or retract the continuum robot 100 into a target structure by advancing, retarding and/or retracting the base stage 52.

An operational console 50 (FIG. 1) may indicate a driving amount to the base stage 52 and, independently, to the actuator 2. The operational console 50 may include dedicated hardware including a field-programmable gate array (FPGA) and the like; and/or may be a computer including a storage unit, a work memory, and a central processing unit (CPU). Where the operational console 50 is a computer, the storage unit may be a memory that stores a software program corresponding to a control system algorithm and the CPU may expand the program in the work memory, and may execute the program line by line, for the computer to function as the operational console 50. In either case, the operational console 50 may communicably connect with the base stage 52 and the actuator 2, and the operational console 50 may send signals representing the driving amount and configuration to these control targets, which may be imputed by an end user through push buttons, joystick or the like. Thus, the continuum robot 100 includes at least one distal bending section 102, with robotic control for insertion and removal of the continuum robot 100 from the target for operation during lung biopsies, medical procedures, and similar operations.

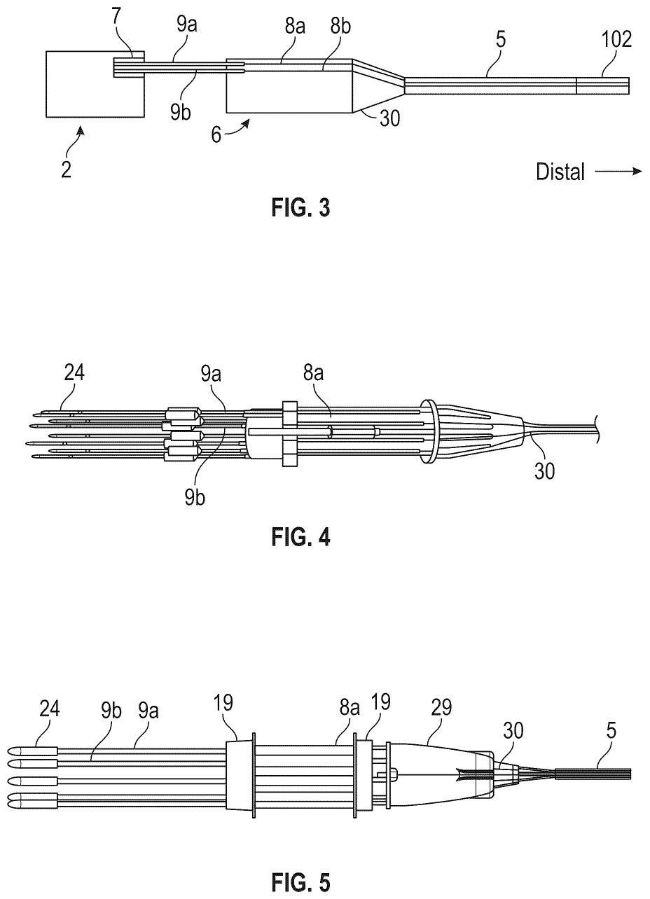

FIG. 3 illustrates an actuator and hub assembled onto a shaft of the continuum robot. As illustrated in FIG. 3, a distal end of the hub body 6 may abut the shaft 5, and a proximal end of the hub body 6 may extend towards the controller 2. A proximal end of the hub body 6 aligns with the actuator clamps 7 of the controller and a distal end of hub body aligns with a diameter of the catheter shaft 5. The changes between such diameters may be referred to as a diameter transition.

At a distal end of a hub cone 30, drive wires 4 converge to a reduced diameter surrounding a tool channel. The drive wires 4 extend from the hub body 6 through the catheter shaft 5, which may have a 4 mm outer diameter (OD).

The catheter shaft 5 may include nine nitinol drive wires 4 with 0.0095″ OD. The longitudinal member, also referred to as hub hypotubes 8a, 8b, may be 304 stainless steel, 26 TW hypotubes, with 0.012″ inner diameter (ID)/0.018″ OD. The tool channel may be a single lumen 63D Pebax© extrusion, 0.091″ ID/0.104″ OD. The catheter shaft 5 may be a multi-lumen 72D Pebax© extrusion, 0.101″ ID/0.1461″ OD, with eighteen small lumens (nine used for drive wires) and a central lumen for tool passage via the tool channel. A proximal catheter shaft lumen guide may be 0.0165″ ID, and 5 mm length. Distal lumens of the catheter shaft 5 may be 0.0125″ ID, extending longitudinally through the distal bending section 102. The hub hypotubes 8a, 8b may be inserted 5 mm into the proximal catheter shaft lumen guide. The tool channel may be inserted into the catheter shaft 5, ending 3 mm past the proximal edge of a distal end of the catheter shaft 5.

Each drive wire 4 may be surrounded and protected by a respective longitudinal member, which similarly extends across the hub body 6 to the catheter shaft 5. The longitudinal members may be formed of a rigid material, e.g., 304 SS 26 TW SS, with a 0.012″ ID and a 0.018″ OD, with a length that may be at least 120 mm.

The drive wires may be anchored at predetermined locations on or within the shaft 5, with each drive wire otherwise being slidable within the respective longitudinal member. The drive wires may be configured to be pushed/pulled with a +/−16 mm stroke length with up to 20 N force. The diameter transition from the catheter shaft to the actuator 2 may be 3.1 mm to 22 mm. Hub hypotubes 8 may buckle within an unsupported section(s), for small diameter hypotubes and long push stroke.

Posture and/or pose of the catheter shaft 5 may be controlled by push/pull on a proximal end of at least one drive wire, which slidably travels within the respective longitudinal members 8a, 8b. The catheter shaft 5 may extend along a longitudinal (X) axis with at least one bending section 102 provided at a distal end thereof. At least three drive wires may terminate on or anchor within the distal bending section 102. The actuator 2 may selectively push/pull drive wires to control posture and/or pose of the distal bending section 102. Pusher rods 9a, 9b, which may be 304 SS 21 RW, 0.020″ ID/0.032″ OD, 61 mm length, may extend from a proximal end of the catheter hub to the actuator, for detachably attaching the driving wires to removably attach the hub body 6 to the actuator 2 and controller (operation console 50).

As shown in FIG. 3, a straight section is provided at a proximal end of the hub body 6, with a diameter corresponding to the proximal end of the diameter transition, where the longitudinal members may terminate for connection of the drive wires to a push/pull assembly of the controller.

FIG. 4 is a perspective view of components within a hub assembly and related components.

As shown in FIG. 4, pusher rods 9 may attach to proximal end of respective driving wires 4 extending from a proximal side of the hub assembly to respective clamp rods 23. The drive wires 4 follow a path corresponding to a slope of the diameter transition of the hub cone 30, which may be molded 72D Pebax©.

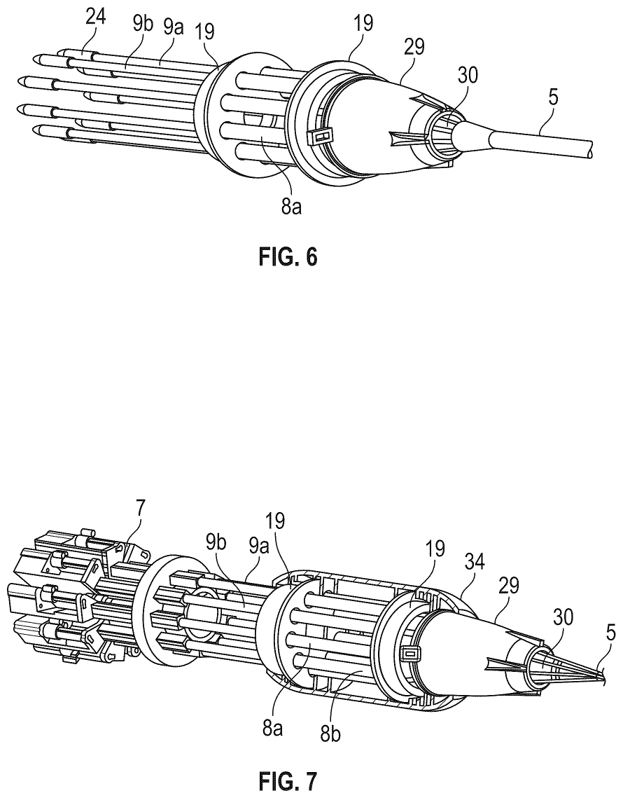

FIG. 5 is a profile view illustrating components within the catheter hub and related components. FIG. 6 is perspective view of the components within the catheter hub and related components. FIG. 7 is a perspective view illustrating connection of proximal components of the catheter hub affixed in respective clamps of a controller.

As illustrated in FIGS. 5 to 7, a plurality of single lumen longitudinal members 8a, 8b and guide disks 19 may be provided. A tool channel may exit between the hub guide discs 19. The catheter shaft 5 may include a central lumen for tool passage through a working length thereof. Pusher rods 9a, 9b may be affixed to the controller via respective clamps 7. The hub cone 30 may be bonded onto the catheter shaft 5 with the longitudinal members 8a, 8b transitioning therebetween. A proximal end of the catheter shaft 5 may include lumens that are larger than lumens on the distal end of catheter shaft 5. The larger lumens on the proximal end of the catheter shaft 5 are sized for an OD of the hub hypotube 8 housed therein. Distal ends of the longitudinal members 8a, 8b may be inserted into proximal ends of catheter shaft lumens. The smaller lumens on the distal end of catheter shaft 5 are sized for an ID of the drive wire 4 housed therein.

A cone cover 29 may be provided to cover at least a part of the hub cone 30 and may also cover a proximal end of the catheter shaft 5. A distal end of the cone cover 29 may fit closely over the OD of the catheter shaft 5. The proximal end of the catheter shaft 5 may have lumens 20 sized to accommodate an OD of a corresponding longitudinal members. The distal end of the longitudinal members 8a, 8b may be inserted into the proximal catheter shaft. The distal end of the tool channel may be inserted into a central tool channel lumen in the catheter shaft 5. The cone cover 29, hub cone 30, tool channel, and catheter shaft 5 may all be formed of thermoplastic materials.

The cone cover 29 may closely fit over at least a first proximal curve on the hub cone 30, to securely press the hub hypotube 8 into a respective groove of a plurality of grooves when the cone cover 29 is affixed thereto. The cone cover 29 may be formed of thermoplastic styrene or other rigid material, e.g., Acrylonitrile Butadiene Styrene (ABS), with an inner diameter of 22.5 mm at the proximal end thereof. The cone cover 29 may be bonded to the hub cone 30 via a wicking adhesive.

An outer shell 34 may be a multi-part shell secured together by screws, snaps, or similar components. When assembled, the outer shell 34 may cover the hub body 6 the assembled hub cone 30 and cone cover 29. The hub cone 30, longitudinal members 8a, 8b, and the catheter shaft extrusions may be compressed together by the assembled outer shell 34. The outer shell 34 may enclose the hub cone 30, the hub cone cover 29, and the plurality of longitudinal members 8a, 8b. When attached to the hub cone 30, the cone cover 29 may press the longitudinal members 8a, 8b against an exterior surface of the hub cone 30.

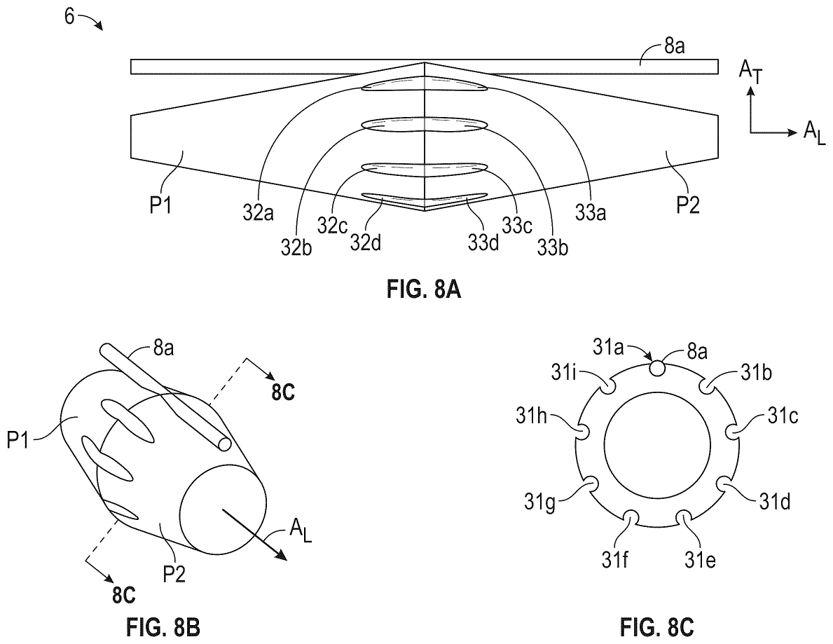

When assembled onto the hub cone 30, the cone cover 29 prevents the longitudinal members 8a, 8b that are supported within plurality of grooves 31a . . . 31h (FIGS. 8A-10) from moving away from/buckling the hub 6 when a pushing force is exerted on an end of one or more of the drive wires 4. A plurality of grooves 31a, 31b, . . . 31e may be extend to corresponding grooves that are formed on the exterior surface of the hub cone 30.

FIG. 8A is a side view of a body supporting a longitudinal member, according to the present disclosure. FIG. 8B is a perspective view of the body of FIG. 8A, according to the present disclosure. FIG. 8C is a sectional view of the body of FIG. 8A, according to the present disclosure.

As shown in FIG. 8A, the body 6 may be formed along a longitudinal axis A_{L} by joining the first part P₁ to the second part P₂. A parting line 650 is formed at the joining of the first part P₁ to the second part P₂, i.e., along the points of contact. The parting line 650 extends along a transverse axis A_{T}, which is perpendicular to the longitudinal axis A_{L}.

As shown in FIG. 8B, a groove, notch or similar configuration may be provided at the first node and/or the second node to maintain the longitudinal member 8a therein.

As shown in FIGS. 8A-8B, the body 6 includes a first part P₁ and a second part P₂. A first plurality of nodes 32a . . . 32i are formed on or through the first part P₁. A second plurality of nodes 33a . . . 33i are formed on or through the second part P₂. A

The first plurality of nodes 32a . . . 32i may be symmetrically arranged along a periphery of the first part P₁, around the longitudinal axis A_{L}. The second plurality of nodes 33a . . . 33i may be symmetrically arranged along a periphery of the second part P₂, around the longitudinal axis A_{L}.

As shown in FIG. 8C, a plurality of grooves 31a . . . 31i are formed by joining the first part P₁ to the second part P₂ and aligning the first plurality of nodes 32a . . . 32i with the second plurality of nodes 33a . . . 33h. Each groove of the plurality of grooves 31a . . . 31i may be configured to receive and support a respective longitudinal member 8a . . . 8i therein. As shown in FIGS. 8A and 8B, a portion of an outer surface of each longitudinal member 8a . . . 8i may protrude from the exterior surface of the body 6.

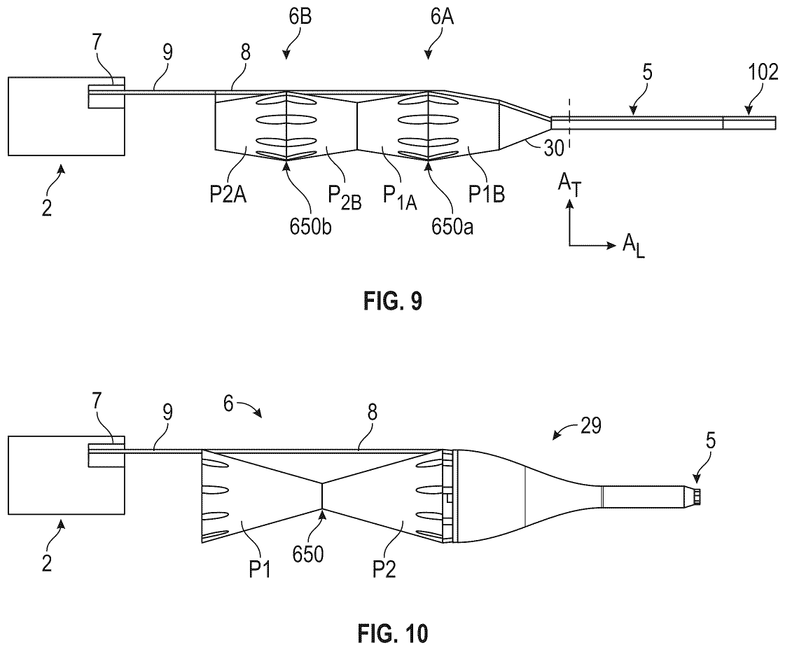

FIG. 9 is a side view of a plurality of bodies providing multiple contact support points for each longitudinal member, according to the present disclosure.

As illustrated in FIG. 9, two bodies 6a, 6b are arranged in series, at least one pusher rod 9 extending from a proximal end for receiving a driving wire 4 extending from a proximal end of at least one respective longitudinal member 8.

Each body of the two bodies 6a, 6b may be arranged as described in FIGS. 8A-8C. For conciseness, the description of each component of the bodies is incorporated by reference herein. As further illustrated in FIG. 9, the two bodies 6a, 6b may be arranged in series between the shaft 5 and controller 2, which are also illustrated and described in detail in the description of FIG. 3. For conciseness, the description of each component of the shaft 5, controller 2 and associated components is incorporated by reference herein.

Each of the first plurality of nodes and second plurality of nodes of each of the first body 6a and the second body 6b are aligned along the longitudinal axis A_{L}. Thus, the longitudinal member 8a extends along the longitudinal axis A_{L} from the shaft 5, across the hub cone 30, across the first body 6a, across the second body 6b, and towards the controller 2. As shown in FIG. 9, a part of the longitudinal member 8a is supported by a groove formed at the parting line 650a of the first body 6a with another proximal part of the first longitudinal member 8a being supported by a groove formed at the parting line 650b of the second body 6b, thereby supporting the longitudinal member 8a as a straight beam that extends from a controller, across the first body 6a, and across the second body 6b.

Additional longitudinal members may similarly extend from the shaft 5 and the hub cone 30, crossing the first body 6a and the second body 6b, and towards the controller 2 in a straight orientation, supported in respective grooves formed at the respective parting line of the first body 6a and the second body 6b. The first body 6a and the second body 6b are axially positioned and are configured to support each longitudinal member of at least nine longitudinal members 8b . . . 8i in respective grooves formed by alignment of respective nodes at the respective parting lines.

As shown in FIGS. 8A-9, the grooves of each of the first body 6a and the second body 6b may be symmetrically arranged along a periphery of the first body 6a and the second body 6b between the controller 2 and shaft 5 of the continuum robot. Thus, each of the first body 6a and the second body 6b rigidly support each of a plurality of supports, i.e., at least nine guide channels, to guide and straighten each longitudinal support of the plurality of supports. The split arrangement and construction of the first body 6a and the second body 6b along respective parting lines provides enhanced rigidity of each of the plurality of longitudinal members.

Each longitudinal member of the plurality of longitudinal members may be supported by each of the first body 6a and the second body 6b. Each longitudinal member of the plurality of plurality of longitudinal members is supported by a respective contact point of the first body 6a and a respective contact point of the second body 6b. The contact points of the first body 6a and the second body 6b are positioned where the nodes of the first body 6a align with the nodes of the second body 6b. Each contact point is provided an outer circumference of the parting line 650, forming a grove 31 along the longitudinal axis A_{L}. The arrangement of the plurality of groves provides a strong and stable connection for the respective supports and related components.

FIG. 10 is a side view of a plurality of bodies supporting a longitudinal member, according to the present disclosure.

As shown in FIG. 10, the cone cover 29 may cover the hub cone 30, and the longitudinal members 8a, 8b may extend from the controller 2 to the proximal end of catheter shaft 5. A multi-part body 6 is provided for supporting a plurality of longitudinal members 8, with the multi-part body 6 including a first part P₁ and a second part P₂. As discussed herein a first plurality of nodes may be formed on or through the first part P₁, surrounding a longitudinal axis A_{L}, and a second plurality of nodes 33 may be formed on or through the second part P₂, also surrounding the longitudinal axis A_{L}. A plurality of grooves 31a . . . 31h may be formed along a longitudinal axis A_{L}. In contrast to the embodiment of FIG. 9, at the distal end the plurality of grooves 31a . . . 31h are formed at an intersection with the hub cone 30 and at the proximal end the plurality of grooves 31a . . . 31h are formed at an intersection with the pusher tubes, with the first part P₁ and the second part P₂ being shaped as one of truncated cones, diamonds or barrels. The first part P₁ is joined to the second part P₂ at a parting line 650 that extends along a transverse axis A_{T}, which is perpendicular to the longitudinal axis A_{L}. Each groove of the plurality of grooves is configured to support a respective longitudinal member of the plurality of longitudinal members 8. Each longitudinal member of the plurality of longitudinal members 8 is configured to slidably house a driving wire of a continuum robot.

In the embodiment of FIG. 10, a smaller base of the first part P₁ is joined to a smaller base of the second part P₂.

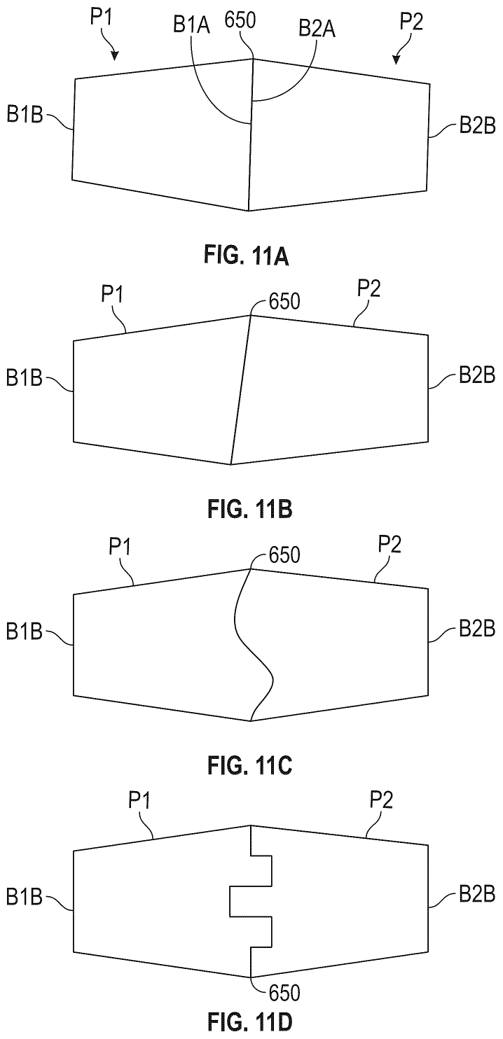

FIGS. 11A to 11D are side views of a first part and a second part of a multi-part body, according to the present disclosure.

In FIGS. 11A to 11D, the first part P₁ and the second part P₂ are shaped as truncated cones, with the parting line 650 therebetween, with a first base B_{1A} of the first part P₁ having a diameter that is substantially identical to a diameter of a first base B_{2A} of the second part P₂, and a second base B_{1B} of the first part P₁ having a diameter that is substantially identical to a diameter of a second base B₂₃ of the second part P₂.

In FIG. 11A, a straight parting line 650 separates the first part P₁ and the second part P₂.

In FIG. 11B, an angled parting line 650 separates the first part P₁ and the second part P₂.

In FIG. 11C, a curved parting line 650 separates the first part P₁ and the second part P₂.

In FIG. 11D, a zigzag parting line 650 separates the first part P₁ and the second part P₂.

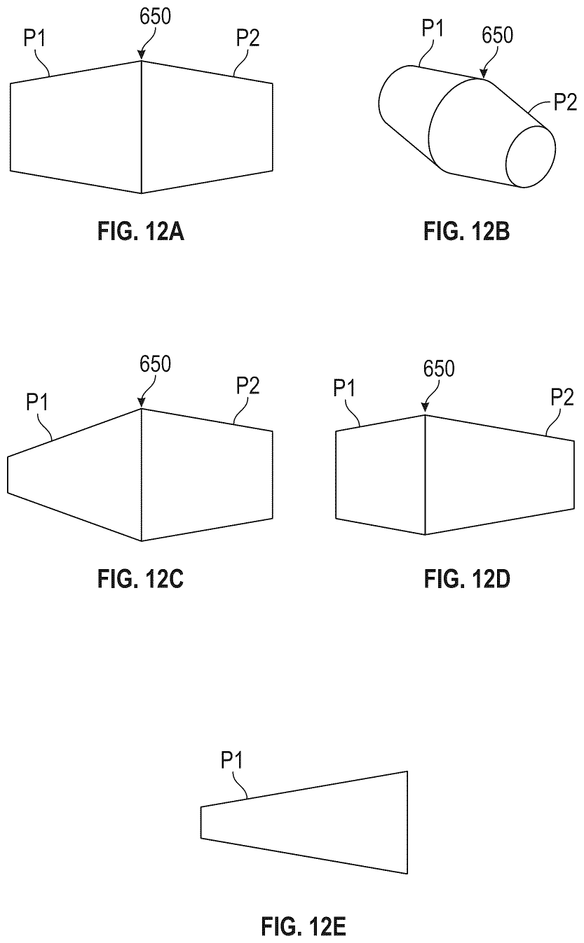

FIGS. 12A to 12E illustrate various parting line positions and varied sizing of the first part and the second part of the multi-part body, according to the present disclosure.

FIGS. 12A and 12B are side and perspective views, respectively, of the first part P₁ and the second part P₂. In FIGS. 12A and 12B, the straight parting line 650 separates the first part P₁ and the second part P₂, which each have a first base with identical diameters and each have a second base with identical diameters. The first part P₁ and the second part P₂ are symmetrical, with similar length.

FIG. 12C is a side view of a first part P₁ and a second part P₂ of similar length. As shown in FIG. 12C, a second base of the first part P₁ has a smaller diameter that a diameter of a second base of the second part P₂. Thus, the first part P₁ and the second part P₂ are of similar length, but are not symmetrical.

In FIG. 12D is a side view of a first part P₁ and a second part P₂ of different lengths, and with a second base of the first part P₁ having a larger diameter that a diameter of a second base of the second part P₂. Thus, the first part P₁ and the second part P₂ are not of similar length, and are not symmetrical.

FIG. 12E is a side view of a first part with an extended length, according to the present disclosure. As shown in FIG. 12E, the first part P₁ has an extended length and a large difference between a diameter of the first base and the second base, thereby providing an extended draft angle, i.e., a difference between diameters of the first base and the second base relative to the length of the truncated cone.

The first part and the second part illustrated in any of FIGS. 12A and 12B may be used to form the multi-part body of the present disclosure.

The first part P₁ and the second part P₂ form a molded part with a parting line that joints the two parts, as effective halves of the molded part. Each of the first part P₁ and the second part P₂ has tapered sides with a draft angle, that may be 0.1 degrees to 10 degrees. Each of the first part P₁ and the second part P₂ may have a different draft angle.

The parting line may be located at varied positioned with respect to the first part P₁ and the second part P₂, and the first part P₁ and the second part P₂ need not have to be of equal size or shape. Each of the first part P₁ and the second part P₂ may be v shaped with the parting line joining the first part P₁ and the second part P₂. The entire whole molded part may have a diamond or similar shape with the parting line at the widest section of the diamond. The parting line may be the contact point for the longitudinal members, i.e., support sleeves, and the parting line may be provided at an end position for parts that require the most support.

Angles between 0.5 degrees and 10 degrees provide consistent manufacturing consistency and yield, with higher draft angles simplifying part remove from the mold without damage to the part or mold. The surface of the part may be textured. Axially positioning in the mold allows support for each of a plurality of longitudinal members while allowing for draft on both sides so the part can be removed easily from the mold. Providing adequate draft allows for parts molding without damage and supporting molding of the first part and the second with the plurality of longitudinal members without side action in the mold, which may be complex and costly.

Each of the first part P₁ and the second part P₁ of the first body 6a and the second body 6b may be molded parts configured to be produced from a single mold. Each of the first part P₁ and the second part P₂ may be molded using a same mold, with each of the first part P₁ and the second part P₂ having substantially identical predetermined configurations. Using substantially identical predetermined configurations of molded part joined at a parting line is usable in various applications, including medical devices and automation. In addition, respective parting lines are positioned substantially at a middle of each of the first body 6a and the second body 6b. The parting lines facilitate easy removal from the mold that may form the first body 6a and may form the second body 6b. Also, use of such similarly molded parts allows for secure support and securing of the longitudinal members. The parting line allows for easy removal from the mold. The point contacts form a grove at the parting line, thereby providing a strong and stable connection between the longitudinal members and related parts.

Accordingly, an aspect of the present disclosure provides a multi-part body 6 for supporting a plurality of longitudinal members 8, with the multi-part body 6 including a first part P₁; a first plurality of nodes 32 formed on or through the first part P₁, surrounding a longitudinal axis A_{L}; a second part P₂; a second plurality of nodes 33 formed on or through the second part P₂, surrounding the longitudinal axis A_{L}; and a plurality of grooves 31a . . . 31h formed along a longitudinal axis A_{L} by joining the first part P₁ to the second part P₂ and aligning the first plurality of nodes 32 with the second plurality of nodes 33. Each groove of the plurality of grooves is configured to support a respective longitudinal member of the plurality of longitudinal members 8. Each longitudinal member of the plurality of longitudinal members 8 is configured to slidably house a driving wire of a continuum robot. Each of the first part P₁ and the second part P₂ are formed using a same mold, and each of the first part P₁ and the second part P₂ may have an identical predetermined configuration.

The first part P₁ and the second part P₂ may be shaped as one of truncated cones, diamonds or barrels. A diameter of a first base B_{1A} of the first part P₁ may be substantially identical to a diameter of a first base B_{2A} of the second part P₂, a diameter of a second base B_{1B} of the first part P₁ may be substantially identical to a diameter of a second base B₂₃ of the second part P₂, the first base B_{1A} of the first part P₁ may be larger than the second base B_{1B} of the first part P₁, and the first base B_{2A} of the second part P₂ may be larger than the second base B₂₃ of the second part P₂.

Contact between the first part P₁ and the second part P₂ forms a parting line that extends along a transverse axis A_{T}, which is substantially perpendicular to the longitudinal axis A_{L}. The parting line joins one of a first base B_{1A} of the first part P₁ with a first base B_{2A} of the second part P₂, the first base B_{1A} of the first part P₁ with a second base B₂₃ of the second part P₂, a second base B_{1B} of the first part P₁ with the first base B_{2A} of the second part P₂, or the second base B_{1B} of the first part P₁ with the second base B₂₃ of the second part P₂. The parting line 650 may be one or more of a straight line, an angled line, a curved line, a zig-zag line, and a rectangular wave shaped line.

Another aspect of the present disclosure provides a longitudinal support formed by alignment of multi-part bodies, with the longitudinal support including a plurality of multi-part bodies arranged in series along a longitudinal axis, each including a first part with a first plurality of nodes, a second part with a second plurality of nodes. Each first plurality of nodes may be formed on or through the respective first part, surrounding the longitudinal axis. Each second plurality of nodes may be formed on or through the respective second part, surrounding the longitudinal axis. A plurality of grooves may be formed along the longitudinal axis by alignment of respective first and second plurality of nodes of the plurality of multi-part bodies.

Each groove of the plurality of grooves may be configured to support a longitudinal member configured to slidably house a driving wire of a continuum robot. Each of the first and second parts of each of the plurality of multi-part bodies may be configured to be formed using a same mold, with each of the first part P₁ and the second part P₂ having an identical predetermined configuration.

The first and second parts of the plurality of multi-part bodies may be shaped as one of truncated cones, diamonds or barrels. A point of contact of each first part with each second part P₂ may form respective parting lines, which extends along a transverse axis A_{T}.

Each parting line may be one or more of a substantially straight line, a substantially angled line, a substantially curved line, a substantially zig-zag line, and a substantially rectangular wave shaped line.

Another aspect of the present disclosure provides a connector that includes at least one body with a first part with a first plurality of nodes, a second part with a second plurality of nodes, and a plurality of grooves; a hub; and a plurality of longitudinal members. The at least one body may be formed by joining the first part to the second part. The first plurality of nodes may be formed on or through the first part, positioned along a periphery of the first part around a longitudinal axis. The second plurality of nodes may be formed on or through the second part positioned along a periphery of the second part around the longitudinal axis. The plurality of grooves may be formed by aligning the first plurality of nodes with the second plurality of nodes. Each longitudinal member of the plurality of longitudinal members may be supported in a respective groove of the plurality of grooves.

A parting line is formed at the joining of the first part to the second part. The parting line extends along a transverse axis substantially perpendicular to the longitudinal axis, in alignment with the support for each of the plurality of grooves.

The connector may include a hub cover. Each longitudinal member of the plurality of longitudinal members may have an inner diameter substantially equal to or larger than a diameter of a driving wire accommodated therein, with the driving wire being slidable within the respective longitudinal member. The plurality of longitudinal members may extend from a controller, across the at least one body, across the hub, to a distal end of a shaft of a continuum robot. The hub cover may enclose at least part of a surface of the hub with the plurality of longitudinal members and the driving wire therebetween. A screw may be provided to attach the at least one body to the hub cover. The controller may include at least one of an actuator and a handle configured to receive user input for manipulation of the driving wire.

### REFERENCE NUMBERS

| | |
|---|---|
| Actuator | 2 |
| Catheter shaft | 5 |
| Hub body | 6 |
| First body | 6a |
| Second body | 6b |
| Actuator clamp | 7 |
| Longitudinal members | 8 |
| Pusher rod | 9 |
| Hub guide disks | 19 |
| Cone cover | 29 |
| Hub cone | 30 |
| Grooves | 31 |
| First nodes | 32 |
| Second nodes | 33 |
| Outer shell | 34 |
| Medical device system | 40 |
| Positioning cart | 44 |
| Navigation software | 46 |
| Operation console | 50 |
| Base stage | 52 |
| Continuum robot | 100 |
| Distal bending section | 102 |
| Middle bending section | 104 |
| Proximal bending section | 106 |
| Connection portions | 121, 122, 123 |
| Parting line | 650 |
| Longitudinal axis | A_{L} |
| First part | P₁ |
| Second part | P₂ |

In referring to the description, specific details are set forth in order to provide a thorough understanding of the examples disclosed. In other instances, well-known methods, procedures, components and circuits have not been described in detail as not to unnecessarily lengthen the present disclosure.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description to describe one element or feature's relationship to another element(s) or feature(s) as illustrated in the various figures. It should be understood, however, that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly. Similarly, the relative spatial terms “proximal” and “distal” may also be interchangeable, where applicable.

The term about, as used herein means, for example, within 10%, within 5%, or less. In some embodiments, the term about may mean within measurement error.

The terms equal, straight, longitudinal, perpendicular, identical, curved, angled, zig-sag, rectangular, cone shaped, diamond shaped, barrel shaped and the like do not refer to mathematical exactness. Rather, these and similar terms are to be understood to refer to what is possible within ordinary manufacturing confines and tolerances, and observations of an ordinary user.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings herein.

The terminology used herein is for the purpose of describing particular embodiments only and is not intended to be limiting. The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “includes”, “including,” and “containing” are to be construed as open-ended terms (i.e., meaning “including, but not limited to,”) unless otherwise noted. Specifically, these terms, when used in the present specification, specify the presence of stated features, integers, steps, operations, elements, and/or components, but do not preclude the presence or addition of one or more other features, integers, steps, operations, elements, components, and/or groups thereof not explicitly stated. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

It will be appreciated that the methods and compositions of the instant disclosure can be incorporated in the form of a variety of embodiments, only a few of which are disclosed herein. Variations of those embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed:

1. A multi-part body for supporting a plurality of longitudinal members, the multi-part body comprising:
a first part;
a first plurality of nodes formed on or through the first part, surrounding a longitudinal axis;
a second part;
a second plurality of nodes formed on or through the second part, surrounding the longitudinal axis; and
a plurality of grooves formed along a longitudinal axis by joining the first part to the second part and aligning the first plurality of nodes with the second plurality of nodes.

2. The multi-part body of claim 1, wherein each groove of the plurality of grooves is configured to support at least a part of a respective longitudinal member of the plurality of longitudinal members.

3. The multi-part body of claim 1, wherein each longitudinal member of the plurality of longitudinal members is configured to slidably house a driving wire of a continuum robot.

4. The multi-part body of claim 1, wherein each of the first part and the second part are formed using a same mold for symmetry of the first part and the second part.

5. The multi-part body of claim 1, wherein the first part and the second part are each shaped as one of a truncated cone, a diamond or a barrel.

6. The multi-part body of claim 5, wherein:
a diameter of a first base of the first part is identical to a diameter of a first base of the second part,
a diameter of a second base of the first part is identical to a diameter of a second base of the second part,
the first base of the first part is larger than the second base of the first part, and
the first base of the second part is larger than the second base of the second part.

7. The multi-part body of claim 1, wherein contact between the first part and the second part forms a parting line that extends along a transverse axis.

8. The multi-part body of claim 7, wherein the parting line joins one of:
a first base of the first part with a first base of the second part,
the first base of the first part with a second base of the second part,
a second base of the first part with the first base of the second part, or
the second base of the first part with the second base of the second part.

9. The multi-part body of claim 7, wherein the parting line is one or more of a straight line, an angled line, a curved line, a zig-zag line, and/or a rectangular wave shaped line.

10. A longitudinal support comprising:
a plurality of multi-part bodies arranged in series along a longitudinal axis, each including a first part with a first plurality of nodes, a second part with a second plurality of nodes, wherein:
each node of the first plurality of nodes is formed on or through a respective first part, surrounding the longitudinal axis;
each node of the second plurality of nodes is formed on or through a respective second part, surrounding the longitudinal axis; and
a plurality of grooves are formed along the longitudinal axis by aligning first plurality of nodes with the second plurality of nodes of each of the plurality of multi-part bodies.

11. The longitudinal support of claim 10, wherein each groove of the plurality of grooves is configured to support at least a part of a longitudinal member configured to slidably house a driving wire of a continuum robot.

12. The longitudinal support of claim 10, wherein each of the first and second parts of each of the plurality of multi-part bodies is configured to be formed using a same mold.

13. The longitudinal support of claim 10, wherein each of the first and second parts of each of the plurality of multi-part bodies is shaped as one of a truncated cone, a diamond or a barrel.

14. The longitudinal support of claim 10, wherein a parting line is formed between each first part and second part, with each parting line extending along a transverse axis.

15. The longitudinal support of claim 14, wherein the parting line is one or more of a straight line, an angled line, a curved line, a zig-zag line, and/or a rectangular wave shaped line.

16. A connector, comprising:
at least one body including a first part with a first plurality of nodes, a second part with a second plurality of nodes, and a plurality of grooves;
a hub; and
a plurality of longitudinal members, wherein:
the at least one body is formed by joining the first part to the second part,
the first plurality of nodes are formed on or through the first part, positioned along a periphery of the first part around a longitudinal axis,
the second plurality of nodes are formed on or through the second part positioned along a periphery of the second part around the longitudinal axis,
the plurality of grooves are formed by aligning the first plurality of nodes with the second plurality of nodes, and
each longitudinal member of the plurality of longitudinal members is supported in a respective groove of the plurality of grooves.

17. The connector of claim 16, wherein:
a parting line is formed at the joining of the first part to the second part, and
the parting line extends along a transverse axis perpendicular to the longitudinal axis, in alignment with the support for each of the plurality of grooves.

18. The connector of claim 16, further comprising a hub cover, wherein each longitudinal member of the plurality of longitudinal members has an inner diameter larger than a diameter of a driving wire accommodated therein, with the driving wire being slidable within the respective longitudinal member.

19. The connector of claim 16, further comprising a hub cover, wherein the plurality of longitudinal members extend from a controller, across the at least one body, across the hub, to a distal end of a shaft of a continuum robot.

20. The connector of claim 16, further comprising a hub cover configured to enclose at least part of a surface of the hub with the plurality of longitudinal members therebetween.
